(19) United States
(12) Patent Application Publication
(10) Pub. No.: US 2012/0142606 A1
(43) Pub. Date: Jun. 7, 2012

(54) TUMOR TARGETING PEPTIDES, THERAPEUTIC AND DIAGNOSTIC COMPOSITIONS COMPRESSING THE PEPTIDES

(76) Inventors: Ingo Schmidt-Wolf, Bonn (DE); Davorka Messmer, La Jolla, CA (US)

(21) Appl. No.: 13/260,482

(22) PCT Filed: Apr. 1, 2009
(86) PCT No.: PCT/US2009/039197

§ 371 Date: Dec. 22, 2011

(51) Int. Cl. A61K 38/16; G01N 33/574; A61P 35/00; C07K 14/00

(52) U.S. Cl. 514/19.4; 530/300; 530/350; 530/326; 530/402; 530/408; 514/19.3; 514/19.5; 435/7.23

(57) ABSTRACT

The present invention is directed to peptides having affinity for tumor cells. The peptides are useful in pharmaceutical compositions in particular for the treatment of cancer. Further, the peptides are useful in diagnostic compositions, in particular for the diagnosis and imaging of cancer. The peptides according to the present invention are peptides selected from the group consisting of: Z₁-KLAKLAKKLAKLAK-Z₂-LTVXPWY-Z₃, Z₁-KLAKLAKKLAKLAK-Z₂-LTVXP-Z₃, Z₁-KLAKLAKKLAKLAK-Z₂-TVXPW-Z₃, Z₁-KLAKLAKKLAKLAK-Z₂-VXPWY-Z₃, Z₁-KLAKLAKKLA KLAK-Z₂-XPWY-Z₃, Z₁-KLAKLAKKLAKLAK-Z₂-LTVXPW-Z₃, di-, tri-, or multimers of the above sequences, wherein each Z₁, Z₂ and Z₃ independently of one another represents any amino acid sequence of n amino acids, n varying from 0 to 50 and n being identical or different in Z₁, Z₂, and Z₃, and wherein X represents any amino acid, whereby each of the amino acid residues in the above sequences may be independently in either L-form or D-form.

The present invention is directed to peptides having affinity for tumor cells. The peptides are useful in pharmaceutical compositions in particular for the treatment of cancer. Further, the peptides are useful in diagnostic compositions, in particular for the diagnosis and imaging of cancer.

Tumor cells often express proteins or complex carbohydrate as specific tumor markers on their surface. This property of tumor cells gave rise to many attempts for an immunotherapeutic approach to target and kill cancer cells.

Current immunotherapeutic antibody-based methods to target and kill cancer cells use two components: One component recognizes cancer cells and the other component kills cells. However, both components are large molecules and chemically combined in ways that are difficult to standardize and often labile. Also, larger drugs are not as efficient in penetrating tumor masses and are thus less likely to kill all tumor cells. As a consequence, current immunotherapeutic therapies for cancer—though successful for some patients—have significant side-effects and a high number of patients are either non-responsive or develop resistance.

In the search for killing domains useful in such antibody-based methods, proapoptotic peptides have been identified as potentially useful, in particular in form of small amphipathic peptides. Small amphipathic peptides preferentially disrupt negatively charged membranes. Mitochondrial membranes have a high content of anionic phospholipids and a large transmembrane potential, in contrast to plasma membranes, which have a low membrane potential. Thus these peptides preferentially disrupt mitochondrial membranes [de Kroon A I, Dolis D, Mayer A, Lill R, de Kruijff B: Phospholipid composition of highly purified mitochondrial outer membranes of rat liver and Neurospora crassa. Is cardiolipin present in the mitochondrial outer membrane?Biochim Biophys Acta 1325(1):108, 1997; Daum G: Lipids of mitochondria. Biochim Biophys Acta 822(1):1, 1985]. The alpha-helical amphipathic peptide KLAKLAKKLAKLAK (SEQ ID NO:1) has hydrophobic residues distributed on one side of the helical axis and cationic residues on the other. This amphipathic peptide has been linked to anti CD19 antibody and this antibody-peptide conjugate killed B lymphoid lines [Marks A J, Cooper M S, Anderson R J, Orchard K H, Hale G, North J M, Ganeshaguru K, Steele A J, Mehta A B, Lowdell M W, Wickremasinghe R G: Selective apoptotic killing of malignant hemopoietic cells by antibody-targeted delivery of an amphipathic peptide. Cancer Res 65(6):2373, 2005]. It has also been linked to a tumor blood vessel homing motif and showed toxicity to angiogenic epithelial cells and thus anti cancer activity [Ellerby H M, Arap W, Ellerby L M, Kain R, Andrusiak R, Rio G D, Krajewski S, Lombardo C R, Rao R, Ruoslahti E, Bredesen D E, Pasqualini R: Anti-cancer activity of targeted pro-apoptotic peptides. Nat Med 5(9):1032, 1999]. However, tumors smaller than 0.5 cm, do not have blood vessel formation and it is critical to eradicate small tumors as well as micrometastases.

A particular emphasis in recent cancer research has been on breast cancer. Breast cancer is the most common cancer in women in the western world. Breast cancer treatment involves surgical removal of the tumor. Unfortunately, escape of malignant cells from the primary tumor prior to surgery makes this procedure less effective in many cases. Half of the women who do not show metastatic spread outside the breast at time of diagnosis eventually die from disseminating disease. Furthermore, large numbers of animal experiments show that removal of the primary tumor is associated with increased proliferation of smaller tumors and metastases. Thus it is critical to target micrometastases immediately after surgery. In most cases, breast cancer is estrogen dependent. Therefore, surgery is often followed by treatment with estrogen antagonists that inhibit estrogen receptor action. This approach has provided a dramatic reduction in breast cancer mortality. However, this treatment is restricted to women with estrogen receptor-positive breast cancer and a substantial proportion of patients become resistant to endocrine therapies. Ultimately, 40% of the patients still die from the disease [Emens L A, Reilly R T, Jaffee E M: Breast cancer vaccines: maximizing cancer treatment by tapping into host immunity. Endocr Relat Cancer 12(1):1, 2005] (American Cancer Society 2002).

The main goal of cancer therapy is to specifically eradicate cancer cells without affecting normal cells. This requires selective recognition of tumor cells. Though tumor-specific antigens are rare, tumor associated antigens (TAAs) are proteins that are over expressed in tumor cells relative to normal tissue. TAAs are used as targets for mAb-directed therapy. HER2 (neu, c-erbB2) is a member of the epidermal growth factor family of tyrosine kinase receptors [Schechter A L, Stern D F, Vaidyanathan L, Decker S J, Drebin J A, Greene M I, Weinberg R A: The neu oncogene: an erb-B-related gene encoding a 185,000-Mr tumour antigen. Nature 312(5994):513, 1984 ]. Approximately 30% of breast cancer patients show gene amplification of over-expression of HER2 [Slamon D J, Clark G M, Wong S G, Levin W J, Ullrich A, McGuire W L: Human breast cancer: correlation of relapse and survival with amplification of the HER-2/neu oncogene. Science 235(4785):177, 1987; Slamon D J, Godolphin W, Jones L A, Holt J A, Wong S G, Keith D E, Levin W J, Stuart S G, Udove J, Ullrich A, et al.: Studies of the HER-2/neu proto-oncogene in human breast and ovarian cancer. Science 244(4905):707, 1989]. 45-68% of patients with pre-invasive ductal carcinoma in situ are HER2 positive [Boland G P, Butt I S, Prasad R, Knox W F, Bundred N J: COX-2 expression is associated with an aggressive phenotype in ductal carcinoma in situ. Br J Cancer 90(2):423, 2004; Rehman S, Crow J, Revell P A: Bax protein expression in DCIS of the breast in relation to invasive ductal carcinoma and other molecular markers. Pathol Oncol Res 6(4):256, 2000]. Since HER2 over-expression plays an important role in the development of breast cancer and several other cancers [Slamon D J, Godolphin W, Jones L A, Holt J A, Wong S G, Keith D E, Levin W J, Stuart S G, Udove J, Ullrich A, et al.: Studies of the HER-2/neu proto-oncogene in human breast and ovarian cancer. Science 244(4905):707, 1989; Schneider P M, Hung M C, Chiocca S M, Manning J, Zhao X Y, Fang K, Roth J A: Differential expression of the c-erbB-2 gene in human small cell and non-small cell lung cancer. Cancer Res 49(18):4968, 1989; Park J B, Rhim J S, Park S C, Kimm S W, Kraus M H: Amplification, overexpression, and rearrangement of the erbB-2 protooncogene in primary human stomach carcinomas. Cancer Res 49(23):6605, 1989], it is an attractive target for antibody-directed therapies. The first monoclonal antibody approved for the use in solid tumor therapy has been Herceptin, a humanized mAb specific for HER2. This mAb is currently used for treatment of breast cancer. HER2 overexpression occurs in ˜20% of breast cancer patients. HER2 overexpression correlates with poor prognosis, chemoresistance, and aggressive and metastatic tumor growth. Only 20-30% of HER2 positive patients respond to Herceptin [Slamon D J, Leyland-Jones B, Shak S, Fuchs H, Paton V, Bajamonde A, Fleming T, Eiermann W, Wolter J, Pegram M, Baselga J, Norton L: Use of chemotherapy plus a monoclonal antibody against HER2 for metastatic breast cancer that overexpresses HER2. N Engl J Med 344(11):783, 2001; Vogel C L, Cobleigh M A, Tripathy D, Gutheil J C, Harris L N, Fehrenbacher L, Slamon D J, Murphy M, Novotny W F, Burchmore M, Shak S, Stewart S J, Press M: Efficacy and safety of trastuzumab as a single agent in first-line treatment of HER2-overexpressing metastatic breast cancer. J Clin Oncol 20(3):719, 2002; Hinoda Y, Sasaki S, Ishida T, Imai K: Monoclonal antibodies as effective therapeutic agents for solid tumors. Cancer Sci 95(8):621, 2004; Pegram M D, Lipton A, Hayes D F, Weber B L, Baselga J M, Tripathy D, Baly D, Baughman S A, Twaddell T, Glaspy J A, Slamon D J: Phase II study of receptor-enhanced chemosensitivity using recombinant humanized anti-p185HER2/neu monoclonal antibody plus cisplatin in patients with HER2/neu-overexpressing metastatic breast cancer refractory to chemotherapy treatment. J Clin Oncol 16(8):2659, 1998]. In addition, severe side-effects like cardiac dysfunction have been observed. The incidence and severity of these was greatest in patients receiving herceptin and anthracycline plus cyclophosphamide [Seidman A, Hudis C, Pierri M K, Shak S, Paton V, Ashby M, Murphy M, Stewart S J, Keefe D: Cardiac dysfunction in the trastuzumab clinical trials experience. J Clin Oncol 20(5):1215, 2002]. A drawback for the clinical use of monoclonal antibodies is the difficulty and expense of commercial scale production and the limited ability to penetrate solid tumors [LeSauteur L, Cheung N K, Lisbona R, Saragovi H U: Small molecule nerve growth factor analogs image receptors in vivo. Nat Biotechnol 14(9):1120, 1996]. To improve their effectiveness antibodies were initially conjugated to radioisotopes. Currently most immunotoxins contain modified plant, bacteria, or fungi toxins [Arnon R, Sela M: In vitro and in vivo efficacy of conjugates of daunomycin with anti-tumor antibodies. Immunol Rev 62:5, 1982; Wargalla U C, Reisfeld R A: Rate of internalization of an immunotoxin correlates with cytotoxic activity against human tumor cells. Proc Natl Acad Sci USA 86(13):5146, 1989]. The two most commonly used bacterial toxins used for the development of immunotoxins are diphtheria toxin and Pseudomonas exotoxin (PE) [Siegall C B: Targeted toxins as anticancer agents. Cancer 74(3 Suppl):1006, 1994]. However, monoclonal antibodies conjugated to bacteria toxins show poor cellular uptake and low penetration into tumor tissue [Aina O H, Sroka T C, Chen M L, Lam K S: Therapeutic cancer targeting peptides. Biopolymers 66(3):184, 2002]. Therefore, smaller fragments like scFv were expected to be more effective. Several clinical trials have been performed using recombinant immunotoxins and they have shown activity in some leukemias and lymphomas [Pastan I: Immunotoxins containing Pseudomonas exotoxin A: a short history. Cancer Immunol Immunother 52(5):338, 2003]. However, there is still no effective therapy for solid tumors. One potential limitation of scFv linked to big bacterial toxins like PE is that it is still a large agent and penetration into solid tumors still poses a challenge. Furthermore, in many cases neutralizing antibodies against the scFv are developed that can interfere with repeated injections which are necessary to reach therapeutic doses. However, another major downside of toxins is that the toxins are conjugated to the antibody and often get cleaved and can cause unspecific toxicity of other tissues including liver.

In view of the above-mentioned prior art it was the object underlying the present invention to provide substances for the targeting of cancer cells which overcome the above-mentioned disadvantages. In particular, it was the object underlying the present invention to provide agents, that can specifically recognize tumor cells and can penetrate the tumor tissue. It was a further object underlying the present invention to provide pharmaceutical and diagnostic compositions for the treatment, diagnosis and/or imaging of cancer.

This problem is solved by peptides selected from the group consisting of:

| | |
|---|---|
| Z₁-KLAKLAKKLAKLAK-Z₂-LTVXPWY-Z₃ | (SEQ ID NO: 2) |
| Z₁-KLAKLAKKLAKLAK-Z₂-LTVXP-Z₃ | (SEQ ID NO: 3) |
| Z₁-KLAKLAKKLAKLAK-Z₂-TVXPW-Z₃ | (SEQ ID NO: 4) |
| Z₁-KLAKLAKKLAKLAK-Z₂-VXPWY-Z₃ | (SEQ ID NO: 5) |
| Z₁-KLAKLAKKLAKLAK-Z₂-XPWY-Z₃ | (SEQ ID NO: 6) |
| Z₁-KLAKLAKKLAKLAK-Z₂-LTVXPW-Z₃ | (SEQ ID NO: 7) |

and di-, tri-, or multimers of the above sequences
   wherein each Z₁, Z₂ and Z₃ independently of one another represents any amino acid sequence of n amino acids, n varying from 0 to 50 and n being identical or different in Z₁, Z₂, and Z₃, and wherein X represents any amino acid, whereby each of the amino acid residues in the above sequences may be independently in either L-form or D-form.

The central feature of the peptides according to the present invention is that they are comparatively small molecules with a limited number of amino acids that comprise two functional domains, namely a breast cancer binding domain and an apoptosis inducing domain.

It has surprisingly be found that the peptides according to the present invention efficiently penetrate cancer tissue due to their small size and kill cancer cells via their apoptosis inducing domain. Another major advantage of these targeted small peptide toxins is that they can be synthesized as one piece, no conjugation is required, thus a more controlled and less toxic product can be synthesized. Furthermore, since they are small peptides, the production is cost effective and thus available to all patients. This proposal has a significant impact on the therapy of cancer establishing the anti-cancer activity of peptide-toxins.

As outlined above, the peptides according to the present invention consist of at least two functional domains, namely a targeting domain that targets the peptide specifically to cancer cells and a killing domain.

Optionally, the peptides according to the invention also contain an N-terminal domain Z1, a linker domain Z2, and/or a C-terminal domain Z3.

A particularly preferred peptide according to the invention is represented by the amino acid sequence Z₁-KLAKLAKKLAKLAK-Z₂-LTVXPWY-Z₃ (SEQ ID NO:2).

If present, the linker domain Z₂ is preferably represented by the amino acid sequence: GG (SEQ ID NO:8).

Several peptides that fall under the general definition of the peptides according to the present invention have been synthesized and tested for their efficiency against cancer. As a result, it was found that the peptide represented by the amino acid sequence KLAKLAKKLAKLAK-GG-LTVXPWY (SEQ ID NO:9) showed particularly advantageous properties.

In a particularly preferred embodiment, the peptide is represented by the amino acid sequence KLAKLAKKLAKLAK-GG-LTVSPWY (SEQ ID NO:10), whereby the amino acids are preferably in L-form. Consequently, the above-mentioned peptide is to be seen as particularly preferred embodiment of the present invention.

The inventors have shown that the peptides according to the present invention can both bind and kill cancer cells in vitro (see Examples). Further, it could be shown that the peptides did not appear to bind immune cells isolated from healthy individuals. As an outcome, a new anti-breast cancer drug with good prospects for clinical utility has been provided. The peptides according to the present invention are significant for treating cancer patients, because they allow a treatment for patients that fail current therapeutic approaches.

In a further preferred embodiment, the peptides according to the present invention are linked with an imaging label. Preferred imaging labels are fluorescent markers and radiomarkers, whereby radiomarkers are particularly preferred.

The present invention also pertains to a pharmaceutical composition comprising at least one of the above-described peptides and, optionally, a pharmaceutically accepted carrier and/or diluent. In this sense, the present invention is also directed to the use of these peptides for the preparation of a pharmaceutical composition for the treatment of cancer.

The pharmaceutical use of the peptides according to the present invention is not limited to any certain type of cancer. However, in a preferred embodiment the peptides according to the present invention are used for the preparation of a pharmaceutical composition for the treatment of breast cancer, prostate carcinoma, colon carcinoma or lymphoma. Most preferably, the peptides according to the present invention are used for the preparation of a pharmaceutical composition for the treatment of breast cancer.

However, the use of these peptides according to the present invention is not limited to therapy. The present invention is also directed to a diagnostic composition comprising at least one peptide according to the invention. Accordingly, the present invention is also directed to the use of the peptides according to the present invention for the diagnosis or imaging of cancer.

Further, the present invention is also directed to a method for the treatment of cancer in a subject comprising administering to the subject an amount sufficient to treat the cancer of a composition comprising at least one of the above-described peptides. In particular, the present invention is directed to such a method for treating breast cancer, prostate carcinoma, colon carcinoma or lymphoma. In a particularly preferred embodiment, the present invention is directed to such a method for the treatment of breast cancer.

The above-mentioned embodiments of the invention will be further elucidated by the following examples, which are to be seen as illustrating the present invention without limiting it.

### EXAMPLE I

### Synthesis of KLAKLAKKLAKLAK-GG-LTVXPWY (all amino acids are in the L-form) (BK1-peptide) (SEQ ID NO:9)

The synthesis of the peptide was conducted according to standard methods well-known in the art.

### EXAMPLE II

### Synthesis of KLAKLAKKLAKLAK-GG-LTVXPWY (SEQ ID NO:9) conjugated to fluoresceinisothiocyanate (FITC) (BK1-FITC)

The synthesis of the conjugated BK1-peptide was conducted as described in the reference Mummert M E, Voss E W Jr., Effects of secondary forces on a high affinity monoclonal IgM anti-fluorescein antibody possessing cryoglobulin and other cross-reactive properties, Mol Immunol. 1998 February; 35(2):103-13.

### EXAMPLE III

### Testing of the Ability of the BK1-FITC Peptide Synthesized in Example II to Target and Kill Cancer Cells

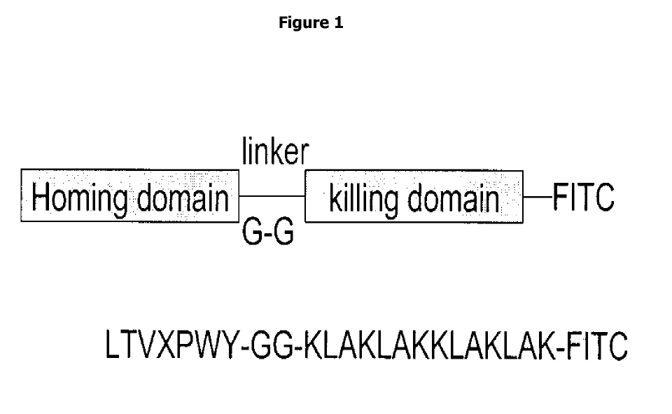

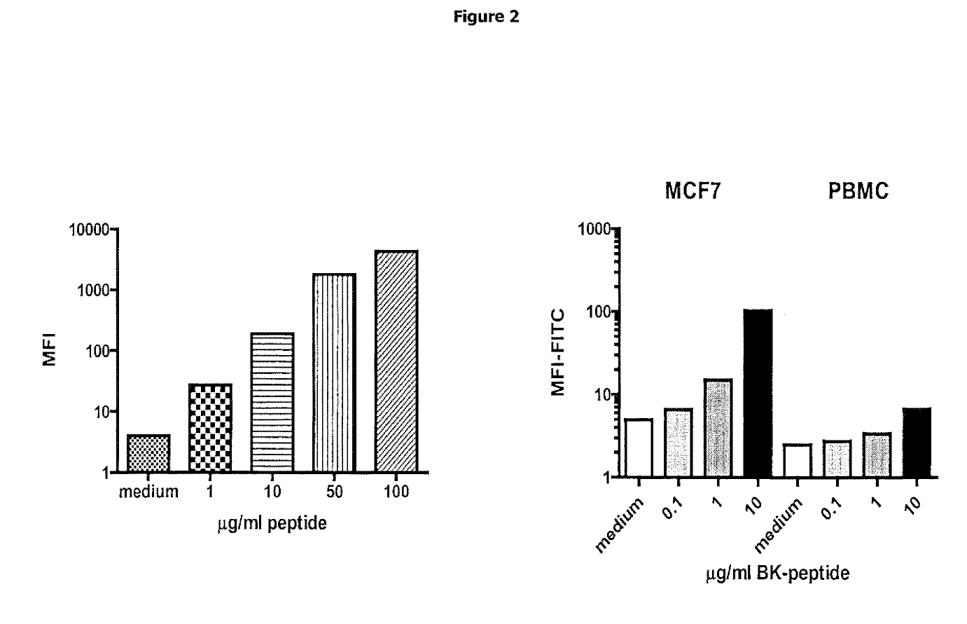

To determine whether the BK-peptide binds to breast cancer cells, we used a breast cancer cell line, MCF7 (purchased from the ATCC # HTB-22). To assess peptide binding, MCF7 cells were incubated with increasing doses (0.1-100 μg/ml) of the BK-FITC peptide at 4° C. for 20 minutes. Cells were subsequently washed 4 times with PBS and analyzed by flow cytometry. A dose dependent increase in mean fluorescence intensity (MFI) was observed, indicating a higher number of peptides bound at higher doses (FIG. 2, left panel). Subsequently, we evaluated the specificity of the BK-peptide for breast cancer cells by incubating MCF7 cells in parallel with human peripheral blood mononuclear cells under the same conditions as mentioned above. At the doses tested we did not observe binding of the peptide to peripheral blood mononuclear cells (FIG. 2, right panel).

FIG. 2. The BK-peptide binds to breast cancer cells (MCF7). MCF7 cells (left panel) and human peripheral blood mononuclear cells (PBMC) (right panel) were incubated with increasing doses of peptide for 20 min at 4° C. The cells were subsequently analyzed by flow cytometry for mean fluorescence intensity of FITC, depicted on the y-axis.

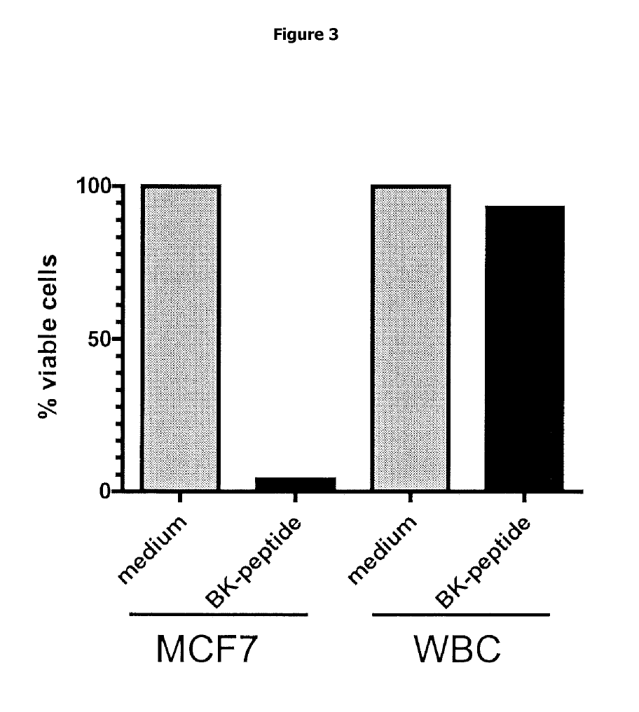

Next, we examined to what extent the peptide specifically kills MCF7 cells. White blood cells (WBCs) were used as a control for normal non-cancerous cells. WBCs were isolated from a healthy donor by ficoll hypaque gradient centrifugation. Red blood cells were removed using a red blood cell lysis buffer and the remaining WBCs were used for the experiment. MCF7 cells and WBCs were incubated with 50 μg/ml peptide or medium only for 24 h. Cells were collected, stained with propidium iodide (PI) and analyzed for apoptosis by flow cytometry (FIG. 3). The data was analyzed by gating on life PI negative cells which is depicted as % viable cells on the y-axis. The data show that under the tested conditions the BK-peptide kills MCF7, but not white blood cells.

1. A peptide selected from the group consisting of:

| | |
|---|---|
| Z₁-KLAKLAKKLAKLAK-Z₂-LTVXPWY-Z₃ | (SEQ ID NO: 2) |
| Z₁-KLAKLAKKLAKLAK-Z₂-LTVXP-Z₃ | (SEQ ID NO: 3) |
| Z₁-KLAKLAKKLAKLAK-Z₂-TVXPW-Z₃ | (SEQ ID NO: 4) |
| Z₁-KLAKLAKKLAKLAK-Z₂-VXPWY-Z₃ | (SEQ ID NO: 5) |
| Z₁-KLAKLAKKLAKLAK-Z₂-XPWY-Z₃ | (SEQ ID NO: 6) |
| Z₁-KLAKLAKKLAKLAK-Z₂-LTVXPW-Z₃ | (SEQ ID NO: 7) |

and di-, tri-, or multimers of the above sequences
wherein each Z₁, Z₂ and Z₃ independently of one another represents any amino acid sequence of n amino acids, n varying from 0 to 50 and n being identical or different in Z₁, Z₂ and Z₃, and
wherein X represents any amino acid; and whereby each of the amino acid residues in the above sequences may be independently in either L-form or D-form.

2. The peptide according to claim 1 represented by the amino acid sequence Z₁-KLAKLAKKLAKLAK-Z₂-LTVXPWY-Z₃ (SEQ ID NO: 2).

3. The peptide according to claim 1, wherein Z₂ represents the amino acid sequence: GG (SEQ ID NO:8).

4. The peptide according to claim 1, represented by the amino acid sequence: KLAKLAKKLAKLAK-GG-LTVXPWY (SEQ ID NO:9).

5. The peptide according to claim 1, represented by the amino acid sequence KLAKLAKKLAKLAK-GG-LTVSPWY (SEQ ID NO: 10), whereby the amino acids used in the sequence are in the L-Form.

6. The peptide according to claim 1, whereby the peptide is linked with an imaging label.

7. The peptide according to claim 6, whereby the imaging label is fluoresceinisothiocyanate.

8. The peptide according to claim 6, whereby the imaging label is a radiomarker.

9. The peptide according to claim 1 for use as a medicament.

10. A pharmaceutical composition comprising at least one peptide according to claim 1 and, optionally, a pharmaceutically acceptable carrier and/or diluent.

11. A method of using a composition according to claim 10 for the preparation of a pharmaceutical composition for the treatment of cancer.

12. The method of claim 11 wherein the cancer is selected from the group consisting of breast cancer, prostate carcinoma, colon carcinoma and lymphoma.

13. The method of claim 11 for wherein the cancer is breast cancer.

14. A method for treating cancer in a subject comprising administering to the subject an amount sufficient to treat the cancer of a composition comprising a compound according to claim 1.

15. The method according to claim 14, whereby the cancer to be treated is selected from the group consisting of breast cancer, prostate carcinoma, colon carcinoma and lymphoma.

16. The method according to claim 15, whereby the cancer to be treated is breast cancer.

17. A diagnostic composition comprising at least one peptide according to claim 1.

18. The use of a peptide according to claim 1 for the diagnosis or imaging of cancer.


SEQUENCE LISTING (10 sequences)

SEQ ID NO: 1 - protein, 14 aa, Artificial Sequence
  Tumor targeting peptide
KLAKLAKKLAKLAK

SEQ ID NO: 2 - protein, 171 aa, Artificial Sequence
  Tumor targeting peptide
XXXXXXXXXXXXXXXXXXXXXXXXXXXXXXXXXXXXXXXXXXXXXXXXXXKLAKLAKKLAKLAKXXXXXXXXXXXXXXXXXXXXXXXXXXXXXXXXXXXXXXXXXXXXXXXXXXLTVXPWYXXXXXXXXXXXXXXXXXXXXXXXXXXXXXXXXXXXXXXXXXXXXXXXXXX

SEQ ID NO: 3 - protein, 169 aa, Artificial Sequence
  Tumor targeting peptide
XXXXXXXXXXXXXXXXXXXXXXXXXXXXXXXXXXXXXXXXXXXXXXXXXXKLAKLAKKLAKLAKXXXXXXXXXXXXXXXXXXXXXXXXXXXXXXXXXXXXXXXXXXXXXXXXXXLTVXPXXXXXXXXXXXXXXXXXXXXXXXXXXXXXXXXXXXXXXXXXXXXXXXXXX

SEQ ID NO: 4 - protein, 169 aa, Artificial Sequence
  Tumor targeting protein
XXXXXXXXXXXXXXXXXXXXXXXXXXXXXXXXXXXXXXXXXXXXXXXXXXKLAKLAKKLAKLAKXXXXXXXXXXXXXXXXXXXXXXXXXXXXXXXXXXXXXXXXXXXXXXXXXXTVXPWXXXXXXXXXXXXXXXXXXXXXXXXXXXXXXXXXXXXXXXXXXXXXXXXXX

SEQ ID NO: 5 - protein, 169 aa, Artificial Sequence
  Tumor targeting peptide
XXXXXXXXXXXXXXXXXXXXXXXXXXXXXXXXXXXXXXXXXXXXXXXXXXKLAKLAKKLAKLAKXXXXXXXXXXXXXXXXXXXXXXXXXXXXXXXXXXXXXXXXXXXXXXXXXXVXPWYXXXXXXXXXXXXXXXXXXXXXXXXXXXXXXXXXXXXXXXXXXXXXXXXXX

SEQ ID NO: 6 - protein, 168 aa, Artificial Sequence
  Tumor targeting peptide
XXXXXXXXXXXXXXXXXXXXXXXXXXXXXXXXXXXXXXXXXXXXXXXXXXKLAKLAKKLAKLAKXXXXXXXXXXXXXXXXXXXXXXXXXXXXXXXXXXXXXXXXXXXXXXXXXXXPWYXXXXXXXXXXXXXXXXXXXXXXXXXXXXXXXXXXXXXXXXXXXXXXXXXX

SEQ ID NO: 7 - protein, 170 aa, Artificial Sequence
  Tumor targeting peptide
XXXXXXXXXXXXXXXXXXXXXXXXXXXXXXXXXXXXXXXXXXXXXXXXXXKLAKLAKKLAKLAKXXXXXXXXXXXXXXXXXXXXXXXXXXXXXXXXXXXXXXXXXXXXXXXXXXLTVXPWXXXXXXXXXXXXXXXXXXXXXXXXXXXXXXXXXXXXXXXXXXXXXXXXXX

SEQ ID NO: 8 - protein, 123 aa, Artificial Sequence
  Tumor targeting peptide
XXXXXXXXXXXXXXXXXXXXXXXXXXXXXXXXXXXXXXXXXXXXXXXXXXKLAKLAKKLAKLAKGGLTVXPWYXXXXXXXXXXXXXXXXXXXXXXXXXXXXXXXXXXXXXXXXXXXXXXXXXX

SEQ ID NO: 9 - protein, 23 aa, Artificial Sequence
  Tumor targeting peptide
KLAKLAKKLAKLAKGGLTVXPWY

SEQ ID NO: 10 - protein, 23 aa, Artificial Sequence
  Tumor targeting peptide
KLAKLAKKLAKLAKGGLTVSPWY